(19) United States
(12) Patent Application Publication
(10) Pub. No.: US 2023/0201497 A1
(43) Pub. Date: Jun. 29, 2023

(54) METHOD FOR PULSATILE DELIVERY OF A GASEOUS DRUG

(71) Applicant: BELLEROPHON THERAPEUTICS, Warren, NJ (US)

(72) Inventor: Martin DEKKER, East Califon, NJ (US)

(73) Assignee: BELLEROPHON THERAPEUTICS, Warren, NJ (US)

(21) Appl. No.: 18/000,188

(22) PCT Filed: May 28, 2021
(86) PCT No.: PCT/US2021/034884

§ 371 Date: Nov. 29, 2022

Related U.S. Application Data
(60) Provisional application No. 63/031,916, filed on May 29, 2020.

(51) Int. Cl. A61M 16/00

(52) U.S. Cl. CPC A61M 16/0057; A61M 16/022

(57) ABSTRACT

Described are methods for providing a pulsed dose of a gaseous drug over a portion of total inspiratory time, where the dose of the gaseous drug is delivered at a concentration of nL of gaseous drug per mL tidal volume.

### CROSS REFERENCE TO RELATED APPLICATIONS

This application claims the benefit of U.S. Provisional Patent Application No. 63/031,916, filed May 29, 2020, entitled “Method for Pulsatile Delivery of a Gaseous Drug,” which is incorporated by reference herein in its entirety.

### FIELD OF THE INVENTION

The present application relates generally to methods for administration of a gaseous drug, in particular, pulsatile delivery of a gaseous drug to patients in need of therapeutic treatment at a concentration based on the tidal volume of the patient.

### BACKGROUND OF THE INVENTION

Nitric oxide (NO) is a gas that, when inhaled, acts to dilate blood vessels in the lungs, improving oxygenation of the blood and reducing pulmonary hypertension. Because of this, nitric oxide is provided as a therapeutic gas in the inspiratory breathing phase for patients having shortness of breath (dyspnea) due to a disease state, for example, pulmonary arterial hypertension (PAH), chronic obstructive pulmonary disease (COPD), combined pulmonary fibrosis and emphysema (CPFE), cystic fibrosis (CF), idiopathic pulmonary fibrosis (IPF), emphysema, interstitial lung disease (ILD), chronic thromboembolic pulmonary hypertension (CTEPH), chronic high altitude sickness, or other lung disease.

Inhaled nitric oxide (iNO) is a well-established safe and effective vasodilator and has been approved for the treatment of persistent pulmonary hypertension in neonates. As disclosed herein, pulse dosing utilizes high concentration pulses to ensure a precise and constant dose regardless of a patient's respiratory rate or inspiratory volume. The pulsatile technology allows titration of the dose, allowing much higher doses/concentrations than currently available in hospital based systems, as well as reduces the overall size of the therapy, allowing it to be administered at home.

While NO may be therapeutically effective when administered under the appropriate conditions, it can also become toxic if not administered correctly. NO reacts with oxygen to form nitrogen dioxide (NO₂), and NO₂ can be formed when oxygen or air is present in the NO delivery conduit. NO₂ is a toxic gas which may cause numerous side effects, and the Occupational Safety & Health Administration (OSHA) provides that the permissible exposure limit for general industry is only 5 ppm. Thus, it is desirable to limit exposure to NO₂ during NO therapy.

Coronaviruses are a family of viruses that can cause varying respiratory illnesses such as the common cold, SARS, and MERS, at various degrees of illness. The SARS-CoV2 virus (also originally known as n-CoV-19), was reported in December 2019 as originating in Wuhan, China, and is a strain of coronavirus that causes coronavirus disease 2019, or COVID-19. Symptoms of SARS-CoV2 infection/COVID-19 include, fever, cough, shortness of breath, and difficulty breathing. Some infected individuals lost the ability to smell and/or taste. Other symptoms may include body aches, pneumonia, chills, fatigue, nausea, diarrhea, and cold-like symptoms such as a runny nose or a sore throat. COVID-19 symptoms can range from mild to severe, and may lead to death, in part, due to complications caused by COVID-19, such as pneumonia and/or organ failure. On the other hand, some people infected with SARS-CoV2 may be asymptomatic. The incubation period for SARS-CoV2 ranges from one to fourteen days, with a median period from five to six days.

The clinical spectrum of the COVID-19 infection ranges from mild signs of upper respiratory tract infection to severe pneumonia and death. Currently, the probability of progression to end stage disease is not well understood; however, preventing progression in patients with mild or moderate disease can likely improve morbidity/mortality and reduce the impact on limited healthcare resources. Furthermore, reducing the need for positive pressure ventilator support as observed in Chen (2004) may limit lung damage. Based on the genomic similarities between the two coronaviruses, the data in SARS-CoV supports the potential for iNO to provide benefit for patients infected with COVID-19. Exogenous iNO in patients who have mild to moderate COVID-19 could prevent further deterioration and potentially improve the time to recovery.

No targeted therapeutic treatments for coronavirus (COVID-19) have been identified. Symptoms range from mild upper respiratory tract infection to severe pneumonia and death. Progression of end stage disease is unpredictable with high fatality rates in mechanically ventilated patients as a result of multi-organ failure. Prevention of COVID-19 progression in spontaneously breathing patients with mild to moderate disease may result in improved morbidity and mortality as well as limiting the burden to limited healthcare resources.

Nitric oxide plays a key role in suppressing viral replication. NO is a naturally produced molecule during the immune response to pathogens, with endogenous NO production upregulated by macrophages as a defense mechanism against some infections including bacterial, viral and protozoal. In vitro studies have shown that NO inhibits the replication of the severe acute respiratory syndrome-related coronavirus (SARS-CoV) (Akerstrom, et al, J. of Virology, 79, 2005, 1966-1969) and improves cellular survival of cells infected with SARS-CoV (Keyaerts et al, Int. J. of Infectious Disease, 8, 2004, 223-226). In a clinical study of SARS patients, iNO demonstrated improvements in arterial oxygenation, reduction in supplemental oxygen and need for ventilatory support. There were also improvements in chest radiography with a reduction in density of lung infiltrates (Chen, et al, Clinical Infectious Disease, 39, 2004, 1531-1535). Although the sample size was small, there appeared to be a shorter time to hospital discharge for those patients in the iNO group compared to controls.

### SUMMARY OF THE INVENTION

In an embodiment of the present invention, a method for delivery of a dose of a gaseous drug to a patient in need is taught. In one embodiment, the method comprises delivering the dose of the gaseous drug to said patient in a pulsatile manner over a portion of the total inspiratory time, wherein the dose of the gaseous drug is delivered at a concentration of nL of gaseous drug per mL tidal volume of the patient.

In an embodiment of the methods of the present invention, the gaseous drug is delivered at a constant rate over a portion of the patient's total inspiratory time. In an embodiment of the methods of the present invention, delivery of the dose of the gaseous drug occurs within the first two-thirds of the total inspiratory time. In an embodiment of the methods of the present invention, delivery of the dose of the gaseous drug occurs within the first half of the total inspiratory time. In an embodiment of the methods of the present invention, delivery of at least fifty percent of the dose of the gaseous drug occurs within the first third of the total inspiratory time. In an embodiment of the methods of the present invention, delivery of at least ninety percent of the dose of the gaseous drug occurs within the first two-thirds of the total inspiratory time. In an embodiment of the methods of the present invention, delivery of at least 70 percent of the dose of the gaseous drug occurs within the first half of the total inspiratory time. In an embodiment of the methods of the present invention, the gaseous drug is delivered in a series of pulses over a period of time. In an embodiment of the methods of the present invention, the gaseous drug delivery has an antimicrobial effect. In an embodiment of the methods of the present invention, the gaseous drug is nitric oxide (NO). In an embodiment of the methods of the present invention, the gaseous drug is carbon monoxide (CO). In an embodiment of the methods of the present invention, the gaseous drug is carbon dioxide (CO₂). In an embodiment of the methods of the present invention, the gaseous drug is heliox (HeO₂). In an embodiment of the methods of the present invention, the gaseous drug is hydrogen sulfide (H₂S). In an embodiment of the methods of the present invention, the portion of inspiratory time is about 0.6 seconds. In an embodiment of the methods of the present invention, the portion of inspiratory time is about 0.4 seconds.

In an embodiment of the invention, a method for treating a viral, bacterial, or protozoal infection in a patient is taught. The method comprises administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient.

In an embodiment of the invention, a method for treating a viral, bacterial, or protozoal infection, which infection leads to development of a disease state in a patient is taught. The method comprises administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the viral, bacterial, or protozoal infection is treated.

In an embodiment of the invention, a method for inhibiting viral, bacterial, or protozoal replication in a patient is taught. The method comprises administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein viral, bacterial, or protozoal replication is inhibited.

In an embodiment of the invention, a method for reducing the need for supplemental oxygen in a patient suffering from a viral, bacterial, or protozoal infection is taught. The method comprises administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the need for supplemental oxygen is reduced or eliminated.

In an embodiment of the invention, a method for improving oxygenation of a patient suffering from viral, bacterial, or protozoal infection is taught. The method comprises administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein oxygenation is improved.

In an embodiment of the invention, a method for improving oxygen saturation of a patient suffering from viral, bacterial, or protozoal infection is taught. The method comprises administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein oxygen saturation is improved.

In an embodiment of the invention, a method for providing supportive care to a patient in respiratory distress due to viral, bacterial, or protozoal infection is taught. The method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the patient's respiratory distress is improved.

In an embodiment of the invention, a method for reducing the time a patient suffering from a viral, bacterial, or protozoal infection is in need of mechanical breathing assistance is taught. The method comprises administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the time in need of mechanical breathing assistance is reduced or eliminated.

In an embodiment of the methods of the present invention, delivery of the dose of nitric oxide occurs within the first half of the total inspiratory time.

In an embodiment of methods of the present invention, the nitric oxide is delivered in a series of pulses over a period of time.

In an embodiment of the methods of the present invention, the nitric oxide is administered in combination with at least one additional gas. In one embodiment, the at least one additional gas is oxygen.

In an embodiment of the methods of the present invention, the method further comprising the administration of at least one additional therapeutic agent.

In an embodiment of the methods of the present invention, administration of the iNO occurs in an outpatient setting.

In an embodiment of the methods of the present invention, the inhaled nitric oxide is administered for at least 24 hours per day over the course of the treatment period. In one embodiment, the inhaled nitric oxide is administered for least 18 hours per day over the course of the treatment period. In one embodiment, the inhaled nitric oxide is administered for least 12 hours per day over the course of the treatment period. In one embodiment, the inhaled nitric oxide is administered for least 8 hours per day over the course of the treatment period.

In an embodiment of the methods of the present invention, the treatment period is at least twenty-one days. In one embodiment, the treatment period is at least fourteen days. In one embodiment, the treatment period is at least ten days. In one embodiment, the treatment period is at least seven days. In one embodiment, the treatment period is at least five days. In one embodiment, the treatment period is at least three days. In one embodiment, the treatment period is at least two days. In one embodiment, the treatment period is five days or less. In one embodiment, the treatment period is four days or less. In one embodiment, the treatment period is three days or less. In one embodiment, the treatment period is two days or less. In one embodiment, the treatment period is one day or less.

In an embodiment of the invention, the viral infection is SARS-CoV2 and the disease state is COVID-19. In an embodiment of the invention, the viral infection is selected from influenza, adenoviruses, parainfluenza viruses, respiratory syncytial virus (RSV), bocavirus, coronaviruses, human metapneumovirus, rhinoviruses and enteroviruses. In an embodiment, the bacterial infection is selected from S. pneumoniae, S. pyogenes, S. aureus, H. influenzae, Bordetella pertussis, Moraxella catarrhalis, Mycoplasma pneumoniae, Mycoplasma hominis, Chlamydia spp, Legionella, Francisella, Yersinia, Coxiella burnetti. Corynebacterium diphtheriae, Corynebacterium haemolyticum, Neisseria gonorrhoeae, and Candida albicans. In an embodiment, the protozoal infection is Toxoplasma gondii.

Various embodiments are listed above and will be described in more detail below. It will be understood that the embodiments listed may be combined not only as listed below, but in other suitable combinations in accordance with the scope of the invention.

The foregoing has outlined rather broadly certain features and technical advantages of the present invention. It should be appreciated by those skilled in the art that the specific embodiments disclosed may be readily utilized as a basis for modifying or designing other structures or processes within the scope present invention. It should also be realized by those skilled in the art that such equivalent constructions do not depart from the spirit and scope of the invention as set forth in the appended claims.

### BRIEF DESCRIPTION OF THE DRAWINGS

The foregoing summary, as well as the following detailed description of the invention, will be better understood when read in conjunction with the appended drawings.

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a comparison between the delivery of NO using an embodiment of the methods of the present invention with iNOpeak pulmonary delivery and the current delivery method of inhalation of a drug laden gas delivered at 160 ppm (constant). As shown, the iNOpeak delivers a constant rate of NO over a portion of the inspiration time, shown as a “square pulse” rather than the traditional smooth wave.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

### Definitions

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The disease state of “interstitial lung disease” or “ILD” shall include all subtypes of ILD, including, but not limited to, idiopathic interstitial pneumonia (IIP), chronic hypersensitivity pneumonia, occupational or environmental lung disease, idiopathic pulmonary fibrosis (IPF), non-IPF IIPs, granulomoutus (e.g., sarcoidosis), connective tissue disease related ILD, and other forms of ILD.

When ranges are used herein to describe an aspect of the present invention, for example, dosing ranges, amounts of a component of a formulation, etc., all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Effective dosing of a gaseous drug is based on a number of different variables, including quantity of drug and the timing of delivery. Current delivery of inhaled drugs includes administering to the patient a drug laden inhaled gas. However, because the patient typically inhales only a fraction of this drug laden gas, a significant amount of drug is wasted as a result. Other current methods of inhaled drug delivery include pulsatile gaseous delivery, which aims to achieve the delivery of a fixed amount of drug into the periphery per hour based on the patient's ideal body weight, which is mathematically derived from the patient's sex and gender.

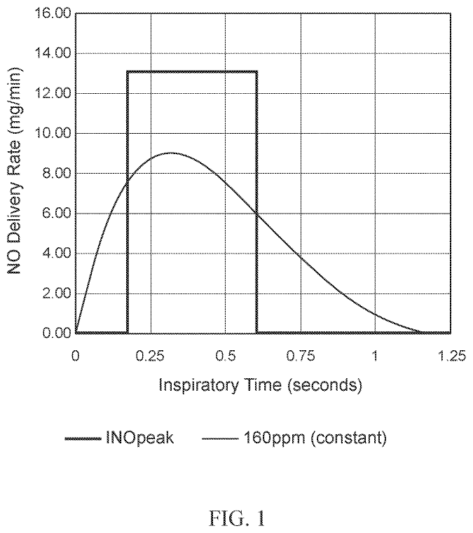

Methods for gaseous drug delivery that provide inhaled drug delivery over current methods are disclosed herein. In one aspect, the present invention includes methods for delivering a dose of a gaseous drug to a patient during the inhalation phase of the breath, wherein the concentration of the dose is based on the tidal volume of the patient. As used herein, the term “tidal volume” refers to the volume of air inhaled and/or exhaled during normal respiration. By administering the dose of gaseous drug at a concentration based on the tidal volume, a fixed amount of the drug is delivered to the patient per breath, providing enhanced drug delivery over other methods that deliver a fixed amount of the drug over a time period, such as a fixed amount of drug per hour. FIG. 1 illustrates the delivery rate of NO using the methods described herein when compared to current delivery systems using the inhalation of a drug laden gas.

Any method for determining tidal volume is contemplated within the present disclosure, as would be understood by one of ordinary skill in the art. In some embodiments, the patient's tidal volume is determined by direct measurement. For example, tidal volume can be measured using spirometry, a pneumotachometer or thermistor cannula positioned in the nose. In some embodiments, the patient's tidal volume is calculated mathematically. For example, tidal volume can be calculated by using a tidal volume proxy, such as through predicted body weight and tidal volume estimation. In some embodiments, the tidal volume ranges from about 200 mL to about 600 mL, about 250 mL to about 550 mL, about 300 mL to about 500 mL, about 300 mL to about 400 mL, about 300 mL, about 400 mL, and about 500 mL.

Any gaseous drug is contemplated for use within the methods of the present disclosure, as would be understood by one of ordinary skill in the art. In some embodiments, the gas is a medical gas. In some embodiments, the gas is a therapeutic gas. In one embodiment, the gaseous drug is nitric oxide. In one embodiment, the gaseous drug is carbon monoxide (CO). In one embodiment, the gaseous drug is heliox (HeO₂). In one embodiment, the gaseous drug is carbon dioxide (CO₂). In one embodiment, the gaseous drug is hydrogen sulfide (H₂S).

### Timing and Delivery of a Pulse of NO

In one aspect, the methods of the invention include the delivery of a dose of a gaseous drug over a specified time frame of the total inspiration time of a single breath.

In an embodiment of the invention, the total inspiratory time refers to the total amount of time that the patient inhales during a single breath. However, depending on context “total inspiratory time” can also refer to a summation of all inspiratory times for all detected breaths during a therapy period. Non-limiting examples of a therapy period include a period of seconds, a period of minutes, and a period of hours. Total inspiratory time may be observed or calculated. In another embodiment, total inspiratory time is a validated time based on simulated breath patterns.

In an embodiment of the invention, the gaseous drug is delivered at a constant rate over a portion of the total inspiratory time. In some embodiments, the portion of inspiratory time during which the drug is delivered ranges from about 0.1 seconds to about 2.0 seconds. In some embodiments, the portion of inspiratory time during which the drug is delivered ranges from about 0.4 seconds to about 0.6 seconds. In one embodiment, the portion of inspiratory time during which the drug is delivered is about 0.4 seconds. In one embodiment, the portion of inspiratory time during which the drug is delivered is about 0.6 seconds.

In one embodiment, a method comprises detecting a breath pattern in a patient. In an embodiment of the invention, the breath pattern includes the total inspiratory time (e.g., the time duration of a single inspiration of a patient). In an embodiment of the invention, the breath pattern is detected using a device comprising a breath sensitivity control. In an embodiment of the invention, the breath pattern is correlated with an algorithm to calculate the timing of administration of a dose of gaseous drug. In an embodiment of the present invention, the volume of gaseous drug containing gas necessary for administration of an amount of gaseous drug on a per pulse basis is calculated. In an embodiment, the gaseous drug is delivered to the patient in a pulsatile manner over a portion of a total inspiratory time. A non-limiting example of a device useful for detecting a breath pattern can be found in PCT Publication No. WO 2016/207227, which is incorporated by reference in its entirety.

In an embodiment of the invention, doses of gaseous drug are delivered to the patient over a period of time sufficient to deliver a therapeutic doses of gaseous drug to the patient. In an embodiment of the invention, the device calculates the total time sufficient to deliver a therapeutic dose of gaseous drug to the patient. In an embodiment of the invention, the total time required for a therapeutic dose of gaseous drug to be delivered to the patient is at least partially dependent upon the breath pattern of said patient.

In some embodiments, the dose of gaseous drug is delivered over a period of time ranging from about 0.03 seconds to about 2.0 seconds. In some embodiments, the gaseous drug is delivered at a constant pulse during each breath. In one embodiment, the dose of gaseous drug is delivered over a period of time ranging from about 0.1 seconds to about 2.0 seconds. In some embodiments, the dose of gaseous drug is delivered over a period of time ranging from about 0.4 seconds to about 0.6 seconds. In one embodiment, the dose of gaseous drug is delivered over a period of time of about 0.4 seconds. In one embodiment the dose of gaseous drug is delivered over a period of time of about 0.6 seconds. In some embodiments, the pulse is duration is adjusted independently.

In an embodiment of the invention, gaseous drug is delivered during the first third of the total inspiratory time. In an embodiment, gaseous drug is delivered during the first half of the total inspiratory time. In an embodiment, gaseous drug is delivered during the first two-thirds of the total inspiratory time.

In an embodiment of the invention, at least fifty percent (50%) of the pulse dose of a gas is delivered over the first third of the total inspiratory time of each breath. In an embodiment of the invention, at least sixty percent (60%) of the pulse dose of a gas is delivered over the first third of the total inspiratory time. In an embodiment of the invention, at least seventy-five percent (75%) of the pulse dose of a gas is delivered over the first third of the total inspiratory time for each breath. In an embodiment of the invention, at least eighty-five (85%) percent of the pulse dose of a gas is delivered over the first third of the total inspiratory time for each breath. In an embodiment of the invention, at least ninety percent (90%) of the pulse dose of a gas is delivered over the first third of the total inspiratory time. In an embodiment of the invention, at least ninety-two percent (92%) of the pulse dose of a gas is delivered over the first third of the total inspiratory time. In an embodiment of the invention, at least ninety-five percent (95%) of the pulse dose of a gas is delivered over the first third of the total inspiratory time. In an embodiment of the invention, at least ninety-nine (99%) of the pulse dose of a gas is delivered over the first third of the total inspiratory time. In an embodiment of the invention, 90% to 100% of the pulse dose of a gas is delivered over the first third of the total inspiratory time.

In an embodiment of the invention, at least seventy percent (70%) of the pulse dose is delivered to the patient over the first half of the total inspiratory time. In yet another embodiment, at least seventy-five percent (75%) of the pulse dose is delivered to the patient over the first half of the total inspiratory time. In an embodiment of the invention, at least eighty percent (80%) of the pulse dose is delivered to the patient over the first half of the total inspiratory time. In an embodiment of the invention, at least 90 percent (90%) of the pulse dose is delivered to the patient over the first half of the total inspiratory time. In an embodiment of the invention, at least ninety-five percent (95%) of the pulse dose is delivered to the patient over the first half of the total inspiratory time. In an embodiment of the invention, 95% to 100% of the pulse dose of a gas is delivered over the first half of the total inspiratory time

In an embodiment of the invention, at least ninety percent (90%) of the pulse dose is delivered over the first two-thirds of the total inspiratory time. In an embodiment of the invention, at least ninety-five percent (95%) of the pulse dose is delivered over the first two-thirds of the total inspiratory time. In an embodiment of the invention, 95% to 100% of the pulse dose is delivered over the first two-thirds of the total inspiratory time. In an embodiment of the invention, 90% to 100% of the pulse dose of a gas is delivered over the total inspiratory time.

When aggregated, administration of a number of pulse doses over a therapy session/timeframe can also meet the above ranges. For example, when aggregated greater than 95% of all the pulse doses administered during a therapy session were administered over the first two thirds of all of the inspiratory times of all of the detected breaths. In higher precision embodiments, when aggregated greater than 95% of all the pulse doses administered during a therapy session were administered over the first third of all of the inspiratory times of all of the detected breaths.

Given the high degree of precision of the detection methodologies of the present invention, a pulse dose can be administered during any specified time window of an inspiration. For example, a pulse dose can be administered targeting the first third, middle third or last third of a patient's inspiration. Alternatively, the first half or second half of an inspiration can be targeted for pulse dose administration. Further, the targets for administration may vary. In one embodiment, the first third of an inspiration time can be targeted for one or a series of inspirations, where the second third or second half may be targeted for one or a series of subsequent inspirations during the same or different therapy session. Alternatively, after the first quarter of an inspiration time has elapsed the pulse dose begins and continues for the middle half (next two quarters) and can be targeted such that the pulse dose ends at the beginning of the last quarter of inspiration time. In some embodiments, the pulse may be delayed by 50, 100, or 200 milliseconds (ms) or a range from about 50 to about 200 milliseconds.

In certain embodiments, the patient or individual can be any age, however, in more certain embodiments the patient is sixteen years of age or older.

### Dosages and Dosing Regimens

In an embodiment of the invention, a dose of gaseous drug delivered to a patient is formulated at a concentration of the amount of gaseous drug per tidal volume of the patient. The dose of gaseous drug to be administered can be calculated based on the amount (such as the weight or volume of the drug) of the drug per unit weight of the patient (for example, mg of drug per kg weight of the patient). In one embodiment, the dose of gaseous drug to be administered is calculated using the following formula:

Target pulse delivery [ml]=Measured Tidal Volume (TV) [ml]×Dose [nl gaseous drug/mL TV]/Concentration [nl gaseous drug/ml]

In one embodiment, a possible tidal volume calculation is as follows:

TV=TV index [ml/kg Predicted Body Weight (PBW)]×PBW [kg];
where TV index is between 4 and 8, and PBW=45.5+2.3*(Height [inch]−60) for female and 50+2.3*(Height [inch]−60) for male.

The amount of gaseous drug can be expressed by weight or by volume.

In an embodiment of the invention, the amount of gaseous drug delivered to the patient is the desired amount of gaseous drug to be administered over the patient's total inspiratory time. The amount of gaseous drug can be expressed by mass or by volume. For example, the amount of gaseous drug can be expressed in mg or nL. As would be understood by one of ordinary skill in the art, an amount of a gaseous drug expressed in a unit of mass can be converted to an amount expressed in a unit of volume, or vice versa, based on the density of the gaseous drug.

In some embodiments, the dose of the gaseous drug is delivered at a concentration of nL of gaseous drug per mL tidal volume. In some embodiments, the dose of the gaseous drug is delivered at a concentration of mg of gaseous drug per mL tidal volume. The gaseous drug may be administered alone or in combination with an alternative gas therapy. In certain embodiments, oxygen (e.g., concentrated oxygen) can be administered to a patient in combination with the gaseous drug. In some embodiments, the dose of the gaseous drug is delivered at a concentration of about 10 nL to about 200 nL of gaseous drug per mL tidal volume.

In some embodiments of the invention, the gaseous drug is nitric oxide. In one embodiment, the amount of nitric oxide delivered to the patient is about 0.001 mg to about 1 mg, about 0.010 mg to about 0.500 mg, about 0.050 mg to about 0.100 mg, or about 0.087 mg. In one embodiment, nitric oxide delivered to a patient is formulated at concentrations of about 0.003 mg to about 0.018 mg NO per mL tidal volume, about 0.006 mg to about 0.010 mg per mL tidal volume, about 0.003 mg NO per mL tidal volume, about 0.006 mg NO per mL tidal volume, or about 0.018 mg NO per mL tidal volume.

In an embodiment of the present invention, a volume of gaseous drug is administered (e.g., in a single pulse) per breath. In some embodiments, the volume of gaseous drug in each pulse dose may be identical during the course of a single session, thus providing the patient with an identical amount of drug per breath. In some embodiments, the volume of gaseous drug in some pulse doses may be different during a single timeframe for gas delivery to a patient.

In an embodiment of the invention, a single pulse dose provides a therapeutic effect (e.g., a therapeutically effective amount of gaseous drug) to the patient. In another embodiment of the invention, an aggregate of two or more pulse doses provides a therapeutic effect (e.g., a therapeutically effective amount of gaseous drug) to the patient.

In an embodiment of the invention, at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 675, about 700, about 750, about 800, about 850, about 900, about 950, or about 1000 pulses of nitric oxide is administered to a patient every hour.

In an embodiment of the invention, a gaseous drug therapy session occurs over a timeframe. In one embodiment, the timeframe is at least about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours per day.

In an embodiment of the invention, a gaseous drug treatment is administered for a timeframe of a minimum course of treatment. In an embodiment of the invention, the minimum course of treatment is about 10 minutes, about 15 minutes, about 20 minutes, about 30 minutes, about 40 minutes, about 50 minutes, about 60 minutes, about 70 minutes, about 80 minutes, or about 90 minutes. In an embodiment of the invention, the minimum course of treatment is about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours. In an embodiment of the invention, the minimum course of treatment is about 1, about 2, about 3, about 4, about 5, about 6, or about 7 days, or about 1, about 2, about 3, about 4, about 5, about 6, about 7, or about 8 weeks, or about 1, about 2, about 3, about 4, about 5, about 6, about 7, about 8, about 9, about 10, about 11, about 12, about 18, or about 24 months.

In an embodiment of the invention, a gaseous drug treatment session is administered one or more times per day. In an embodiment of the invention, gaseous drug treatment session may be once, twice, three times, four times, five times, six times, or more than six times per day. In an embodiment of the invention, the treatment session may be administered once a month, once every two weeks, once a week, once every other day, daily, or multiple times in one day.

### Administration of Oxygen

In an embodiment of the invention, oxygen is administered to the patient in accordance with instructions from a treating physician. In an embodiment of the invention, the oxygen is administered at up to 20 L/minute. In an embodiment of the invention, the oxygen is administered at up to 1 L/minute, 2 L/minute, 3 L/minute, 4 L/minute, 5 L/minute, 6 L/minute, 7 L minute, 8 L/minute, 9 L/minute, 10 L/minute, 11 L/minute, 12 L/minute, 13 L/minute, 14 L/minute, 15 L/minute, 16 L/minute, 17 L/minute, 18 L/minute, 19 L/minute, or 20 L/minute. In an embodiment of the invention, oxygen is administered as prescribed by a physician. In another embodiment, the patient is administered oxygen 24 hours per day. In another embodiment, the patient is administered oxygen for at least 1, 2, 3, 4, 5, 6, 7, 8, 9, 10, 11, 12, 13, 14, 15, 16, 17, 18, 19, 20, 21, 22, 23, or 24 hours per day. In another embodiment, the patient is administered oxygen for at least 12 hours per day.

### Methods of Treatment

In an embodiment of the invention, methods for delivery of a dose of a gaseous drug to a patient in need are taught. In some embodiments, the method includes delivering the dose of the gaseous drug to said patient in a pulsatile manner over a portion of the total inspiratory time, wherein the dose of the gaseous drug is delivered at a concentration of an amount of gaseous drug per tidal volume of the patient. Such delivery is useful for the treatment of various diseases, such as but not limited to idiopathic pulmonary fibrosis (IPF), pulmonary arterial hypertension (PAH), including Groups I-V pulmonary hypertension (PH), chronic obstructive pulmonary disease (COPD), combined pulmonary fibrosis and emphysema (CPFE), cystic fibrosis (CF), emphysema, interstitial lung disease (ILD), chronic thromboembolic pulmonary hypertension (CTEPH), chronic high altitude sickness, or other lung disease, and is also useful as an antimicrobial, for example, in treating pneumonia.

In an embodiment of the invention, methods for treating an infection are taught. In some embodiments, the infection is a viral, bacterial, or protozoal infection. Any viral, bacterial, or protozoal infection is contemplated by the present disclosure. Non-limiting examples of infections include the SARS-CoV2 infection, a P. aeruginosa infection, pneumonia, ventilator-associated pneumonia (VAP), lung infection in cystic fibrosis patients, non-tuberculosis mycobacteria, Mycobacterium Avium Complex (MAC), Mycobacterium abscessus (M abs), SARS-CoV (original SARS), MERS (middle east respiratory syndrome), whooping cough (pertussis), common cold, sinusitis, pharyngitis, epiglottitis, laryngotracheitis, bronchitis, bronchiolitis. In one embodiment, the infection is the SARS-CoV2 infection. Examples of viruses that are capable of causing infections include, but are not limited to, influenza, adenoviruses, parainfluenza viruses, respiratory syncytial virus (RSV), bocavirus, coronaviruses, human metapneumovirus, rhinoviruses and enteroviruses. Examples of bacteria that are capable of causing infections include, but are not limited to, S. pneumoniae, S. pyogenes, S. aureus, H. influenzae, Bordetella pertussis, Moraxella catarrhalis, Mycoplasma pneumoniae, Mycoplasma hominis, Chlamydia spp, Legionella, Francisella, Yersinia, Coxiella burnetti. Corynebacterium diphtheriae, Corynebacterium haemolyticum, Neisseria gonorrhoeae, and Candida albicans. of protozoa that are capable of causing infections include, but are not limited to, Toxoplasma gondii.

In another embodiment, methods for treating symptoms of an infection or disease, a bacterial or viral infection are taught. In one embodiment, the infection is the SARS-CoV2 infection, COVID-19. In another embodiment, methods for improving oxygen saturation in a patient are taught. In another embodiment, methods for improving oxygen saturation of a patient suffering from a viral, bacterial, or protozoal infection are taught. In another embodiment, methods for inhibiting viral, bacterial, or protozoal replication virus in a patient are taught. In another embodiment, method for reducing the need for supplemental oxygen in a patient suffering from a viral, bacterial, or protozoal infection are taught. In another embodiment, methods for improving oxygenation of a patient suffering from a viral, bacterial, or protozoal infection are taught. In another embodiment, methods for providing supportive care to a patient in respiratory distress due to a viral, bacterial, or protozoal infection are taught. In another embodiment, methods for improving oxygenation in a patient are taught. In another embodiment, methods for reducing the requirement for oxygen therapy or reducing the amount of time a patient is on oxygen therapy are taught. In another embodiment, methods for reducing the need for or reducing the amount of time a patient is on mechanical breathing assistance, e.g., a ventilator or intubation, are taught. In another embodiment, methods for reducing the time a patient suffering from a viral, bacterial, or protozoal infection is in need of mechanical breathing assistance are taught. In another embodiment, a method of treating COVID-19 is taught. In another embodiment, a method for reducing the severity of respiratory symptoms associated with COVID-19 is taught. In another embodiment, a method for treating acute respiratory distress syndrome (ARDS) associated with COVID-19 is taught. In another embodiment, methods of use in an outpatient setting are taught.

The methods include administration of a gaseous drug, such as iNO, in accordance with the dosing and dosing regimens discussed herein, and optionally supplementing the gaseous drug administration with oxygen. In an embodiment of the invention, gaseous drug is administered according to the pulsed manner discussed herein. In an embodiment of the invention, the gaseous drug is delivered to a patient using the INOpulse® device (Bellerophon Therapeutics).

In an embodiment of the invention, oxygenation in a patient is improved. In one embodiment, oxygenation is improved as compared with a baseline oxygenation level. In one embodiment, oxygenation is improved by about 1% to about 50%. In another embodiment, oxygenation is improved by about 1% to about 25%. In another embodiment, oxygenation is improved by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, oxygenation is improved by about 10%, 15%, 20%, 25%, 30%, 35%, 40%, 45%, or 50%.

In another embodiment of the invention, oxygenation is maintained as compared with a baseline oxygenation level. In another embodiment, oxygenation is does not decrease as compared with a baseline oxygenation level. In another embodiment, oxygenation declines less over time in treated patients than untreated or placebo patients.

In an embodiment of the invention, oxygen saturation levels are improved. In one embodiment, the oxygen saturation levels are improved as compared with a baseline oxygen saturation level. In one embodiment, oxygen saturation levels are improved by about 1% to about 50%. In another embodiment, oxygen saturation levels are improved by about 1% to about 25%. In another embodiment, oxygen saturation levels are improved by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, oxygen saturation levels are improved by about 10%, 15%, 20%, 25%, 30%, 35%, 40%, 45%, or 50%.

In another embodiment of the invention, oxygen saturation levels are maintained as compared with a baseline oxygen saturation level. In another embodiment, oxygen saturation levels do not decrease as compared with a baseline oxygen saturation level. In another embodiment, oxygen saturation levels decline less over time in treated patients than untreated or placebo patients.

In an embodiment of the invention, the time a patient is on mechanical breathing assistance is reduced as compared to an untreated patient. In one embodiment, the time on mechanical breathing assistance is reduced by about 1% to about 50%. In another embodiment, the time on mechanical breathing assistance is reduced by about 1% to about 25%. In another embodiment, the time on mechanical breathing assistance is reduced by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, the time on mechanical breathing assistance is reduced by about 10%, 15%, 20%, 25%, 30%, 35%, 40%, 45%, or 50%. In another embodiment, treatment with a gaseous drug, such as iNO, according to the present invention avoids the need for mechanical breathing assistance.

In an embodiment of the invention, the time a patient is on supplemental oxygen therapy is reduced as compared to an untreated patient. In one embodiment, the time on supplemental oxygen therapy is reduced by about 1% to about 50%. In another embodiment, the time on supplemental oxygen therapy is reduced by about 1% to about 25%. In another embodiment, the time on supplemental oxygen therapy is reduced by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, the time on supplemental oxygen therapy is reduced by about 10%, 15%, 20%, 25%, 30%, 35%, 40%, 45%, or 50%. In another embodiment, treatment with a gaseous drug, such as iNO, according to the present invention avoids the need for supplemental oxygen therapy.

In an embodiment of the invention, reduction of the severity of respiratory symptoms associated with a viral, bacterial, or protozoal infection and disease states associated therewith, including, for example, SARS-CoV2 and COVID-19, occurs with treatment of a gaseous drug, such as iNO, according to the present invention.

In an embodiment of the invention, the dose of gaseous drug ranges from about 1 nL/mL tidal volume (TV) to about 250 nL/mL TV. In an embodiment of the invention, the dose of gaseous drug ranges from about 100 nL/mL TV to about 200 nL/mL TV.

In an embodiment of the invention, the dose of gaseous drug ranges from about 1 mcg/kg IBW/hr to about 250 mcg/kg IBW/hr. In an embodiment of the invention, the dose of gaseous drug ranges from about 125 mcg/kg IBW/hr to about 250 mcg/kg IBW/hr.

In an embodiment of the invention, the dose of gaseous drug for treating COVID-19 ranges from about 125 mcg/kg IBW/hr to about 250 mcg/kg IBW/hr.

In an embodiment of the invention, the dosing regimen includes administration of a gaseous drug for a period of up to 24 hours daily, for a period of about one day, two days, three days, four days, five days, six days, or seven days, and up to fourteen days, depending on the clinical necessity for the gaseous drug.

In an embodiment of the invention, the dosing regimen is about 125 mcg/kg IBW/hr of iNO for a period of up to 24 hours daily, for a period of about one day, two days, three days, four days, five days, six days, or seven days, or fourteen days, and up to twenty-eight days, depending on the clinical necessity for the iNO.

In an embodiment of the invention, the dosing regimen is about 250 mcg/kg IBW/hr of iNO for a period of up to 24 hours daily, for a period of about one day, two days, three days, four days, five days, six days, or seven days, and up to twenty eight days, depending on the clinical necessity for the iNO.

In an embodiment of the invention, the gaseous drug is administered in an outpatient setting to avoid the need for a patient to be admitted to the hospital, or if already hospitalized, to lessen the time required to be in a hospital setting. Such an outpatient setting can be the patient's home, a clinic, or an ambulatory environment.

### Administration of Other Therapeutic Agents

In an embodiment of the invention, the gaseous drug is administered before, concurrently with, or after, another therapeutic agent. In an embodiment, a therapeutically effective amount of another therapeutic agent is administered to a patient in need thereof to treat a bacterial or viral infection, or a disease caused by such a bacterial or viral infection. In one embodiment, the therapeutic agent is an anti-IL-6 antibody, hydroxychloroquine, chloroquine, favilar, remdesivir, a vaccine, an anti-inflammatory, a steroid (e.g., glucocorticoid such as prednisone, prednisolone, or methylprednisone) or a derivative or precursor thereof. In another embodiment, the therapeutic agent is an agent useful in treating respiratory disease, breathing difficulties, and/or pneumonia.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Calculation of Target Pulse Delivery Volumes

Target pulse volumes for delivery of nitric oxide were calculated based on the measured tidal volume, the desired dose, and a NO concentration of 5000 ppm using the following formula:

Target pulse delivery [ml]=Measured Tidal Volume (TV) [ml]×Dose [nl NO/mL TV]/Concentration [ppm NO]

Table 1 shows examples of the calculated target pulse volumes.

TABLE 1
Calculated Target Pulse volume with a source drug
concentration of 5000 ppm NO, balance Nitrogen

| Measured Tidal | Dose (nl NO/ | Target pulse |
|---|---|---|
| Volume (ml) | ml TV) | volume (ml) |
| 300 | 20 | 1.2 |
| 350 | 160 | 11.2 |
| 400 | 200 | 16 |
| 450 | 120 | 10.8 |
| 500 | 40 | 4 |
| 550 | 10 | 1.1 |

While preferred embodiments of the invention are shown and described herein, such embodiments are provided by way of example only and are not intended to otherwise limit the scope of the invention. Various alternatives to the described embodiments of the invention may be employed in practicing the invention.

We claim:

1. A method for delivery of a dose of a gaseous drug to a patient in need, said method comprising delivering the dose of the gaseous drug to said patient in a pulsatile manner over a portion of the total inspiratory time, wherein the dose of the gaseous drug is delivered at a concentration of nL of gaseous drug per mL tidal volume of said patient.

2. The method of claim 1, wherein the gaseous drug is delivered at a constant rate over a portion of the patient's total inspiratory time.

3. The method of claim 1, wherein delivery of the dose of the gaseous drug occurs within the first two-thirds of the total inspiratory time.

4. The method of claim 1, wherein delivery of the dose of the gaseous drug occurs within the first half of the total inspiratory time.

5. The method of claim 1, wherein delivery of at least fifty percent of the dose of the gaseous drug occurs within the first third of the total inspiratory time.

6. The method of claim 1, wherein delivery of at least ninety percent of the dose of the gaseous drug occurs within the first two-thirds of the total inspiratory time.

7. The method of claim 1, wherein delivery of at least 70 percent of the dose of the gaseous drug occurs within the first half of the total inspiratory time.

8. The method of claim 1, wherein the gaseous drug is delivered in a series of pulses over a period of time.

9. The method of claim 1, wherein the gaseous drug delivery has an antimicrobial effect.

10. The method of claim 1, wherein the gaseous drug is nitric oxide (NO).

11. The method of claim 1, wherein the gaseous drug is carbon monoxide (CO).

12. The method of claim 1, wherein the gaseous drug is carbon dioxide (CO₂).

13. The method of claim 1, wherein the gaseous drug is heliox (HeO₂).

14. The method of claim 1, wherein the gaseous drug is hydrogen sulfide (H₂S).

15. The method of claim 2, wherein the portion of inspiratory time is about 0.6 seconds.

16. The method of claim 15, wherein the portion of inspiratory time is about 0.4 seconds.

17. A method for treating a viral, bacterial, or protozoal infection in a patient, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient.

18. A method for treating a viral, bacterial, or protozoal infection leading to development of a disease state in a patient, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the viral, bacterial, or protozoal infection is treated.

19. A method for inhibiting viral, bacterial, or protozoal replication virus in a patient, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein viral, bacterial, or protozoal replication is inhibited.

20. A method for reducing the need for supplemental oxygen in a patient suffering from a viral, bacterial, or protozoal infection, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the need for supplemental oxygen is reduced or eliminated.

21. A method for improving oxygenation of a patient suffering from a viral, bacterial, or protozoal infection, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein oxygenation is improved.

22. A method for improving oxygen saturation of a patient suffering from a viral, bacterial, or protozoal infection, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein oxygen saturation is improved.

23. A method for providing supportive care to a patient in respiratory distress due to a viral, bacterial, or protozoal infection, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the patient's respiratory distress is improved.

24. A method for reducing the time a patient suffering from a viral, bacterial, or protozoal infection is in need of mechanical breathing assistance, the method comprising administering a dose of a therapeutically effective amount of inhaled nitric oxide to said patient in a pulsatile manner over a portion of the total inspiratory time, wherein the dose is delivered at a concentration of nL nitric oxide/mL tidal volume of said patient, wherein the time in need of mechanical breathing assistance is reduced or eliminated.

25. The method of any of claims 17-24, wherein delivery of the dose of nitric oxide occurs within the first half of the total inspiratory time.

26. The method of any of claims 17-24, wherein the nitric oxide is delivered in a series of pulses over a period of time.

27. The method of any of claims 17-24, wherein the nitric oxide is administered in combination with at least one additional gas.

28. The method of claim 27, wherein the at least one additional gas is oxygen.

29. The method of any of claim 27 or 28, further comprising the administration of at least one additional therapeutic agent.

30. The method of any of claims 17-24, wherein administration of the nitric oxide occurs in an outpatient setting.

31. The method of claims 17-24, wherein the inhaled nitric oxide is administered for at least 24 hours per day over the course of the treatment period.

32. The method of claim 31, wherein the inhaled nitric oxide is administered for least 18 hours per day over the course of the treatment period.

33. The method of claim 32, wherein the inhaled nitric oxide is administered for least 12 hours per day over the course of the treatment period.

34. The method of claim 33, wherein the inhaled nitric oxide is administered for least 8 hours per day over the course of the treatment period.

35. The method of any of claims 31-34, wherein the treatment period is at least twenty-one days.

36. The method of claim 31, wherein the treatment period is at least fourteen days.

37. The method of claim 36, wherein the treatment period is at least ten days.

38. The method of claim 37, wherein the treatment period is at least seven days.

39. The method of claim 38, wherein the treatment period is at least five days.

40. The method of claim 39, wherein the treatment period is at least three days.

41. The method of claim 40, wherein the treatment period is at least two days.

42. The method of any one of claims 17-41, wherein the viral infection is SARS-CoV2 and the disease state is COVID-19.

43. The method of any one of claims 17-41, wherein the viral infection is selected from influenza, adenoviruses, parainfluenza viruses, respiratory syncytial virus (RSV), bocavirus, coronaviruses, human metapneumovirus, rhinoviruses and enteroviruses.

44. The method of any one of claims 17-41, wherein the bacterial infection is selected from S. pneumoniae, S. pyogenes, S. aureus, H. influenzae, Bordetella pertussis, Moraxella catarrhalis, Mycoplasma pneumoniae, Mycoplasma hominis, Chlamydia spp, Legionella, Francisella, Yersinia, Coxiella burnetti. Corynebacterium diphtheriae, Corynebacterium haemolyticum, Neisseria gonorrhoeae, and Candida albicans.

45. The method of any one of claims 17-41, wherein the protozoal infection is Toxoplasma gondii.
